(19) United States
(12) Patent Application Publication
(10) Pub. No.: US 2024/0307396 A1
(43) Pub. Date: Sep. 19, 2024

(54) COMPOSITIONS AND METHODS FOR TREATING CELIAC DISEASE

(71) Applicant: THE REGENTS OF THE UNIVERSITY OF CALIFORNIA, Oakland, CA (US)

(72) Inventor: Guy Weiss, Los Angeles, CA (US)

(21) Appl. No.: 18/566,270

(22) PCT Filed: Jun. 3, 2022
(86) PCT No.: PCT/US22/32114

§ 371 Date: Dec. 1, 2023

Related U.S. Application Data
(60) Provisional application No. 63/197,766, filed on Jun. 7, 2021.
(60) Provisional application No. 63/317,356, filed on Mar. 7, 2022.

(51) Int. Cl. A61K 31/519; A61P 37/08

(52) U.S. Cl. CPC A61K 31/519; A61P 37/08

(57) ABSTRACT

The present disclosure relates to methods for treating celiac disease, especially refractory celiac disease, by administering a therapeutic amount of a JAK inhibitor, or a salt or prodrug thereof, with or instead of gluten-free diet, to a subject in need thereof. Additionally, methods are presented for preventing lymphoma in a subject having or suspected of having celiac disease.

### CROSS-REFERENCE TO RELATED APPLICATIONS

This application claims the benefit of the following U.S. Provisional Application Nos.: 63/197,766, filed Jun. 7, 2021, and 63/317,356, filed Mar. 7, 2022, the entire contents of each are incorporated herein by reference.

### BACKGROUND

Celiac disease (CD) is a common, underdiagnosed gluten associated enteropathy, affecting up to 1% of the US population, currently fully treated only with dietary modifications, and has no other approved therapy. A small subset of patients, likely up to 1.5% of patients, however, proceed to develop the rare refractory CD. Refractory celiac disease (RCD) occurs when patients with confirmed CD have continuous or recurrent malabsorption and enteropathy after at least 12 months on a strict gluten free diet (GFD). Differentiating between Type 1 and Type II RCD is key as the latter is associated with T-cell aberrancy and considered a pre-lymphoma state, with very high mortality rates (Rubio-Tapia et al. (2016) Aliment. Pharmacol. Ther., 44: 704-14; Malamut et al. (2009) Gastroenterology, 136: 81-90; Al-toma et al. (2007) Gut, 56(10):1373-8; Rubio-Tapia et al. (2009) Gastroenterology, 136: 99-107). Identifying this subset of patients is crucial in light of the difference in outcomes between those with Type I RCD and Type II. Type II RCD is defined by the presence of aberrant, clonal intraepithelial T-cell lymphocytes that are not present in Type I RCD (Cellier et al. (2000) Lancet, 356: 203-8). Experimental treatment regimens for Type II RCD include corticosteroids, biologics, and chemotherapy. However, many Type II RCD patients will not respond to these treatments. While Type IRCD is associated with a five-year survival of 80-96% with mortality from malabsorption or non-CD-related causes, the five-year survival of Type II RCD patients is only 44-58%. This lower survival rate is tied to high rates of enteropathy associated T-cell lymphoma (EATL) in Type II RCD ((Rubio-Tapia et al. (2016); Malamut et al. (2009); Al-toma et al.; Rubio-Tapia et al. (2009)).

In view of the foregoing, there is an urgent need for new treatments for patients with Type II RCD, treatments that may also be useful for uncomplicated CD, non-responsive or slow-responsive CD (NRCD/SRCD), and type I RCD, as a substitute or adjunct to the mandatory GFD or other experimental regimens. Dermatitis herpetiformis (DH) and gluten neuropathy/ataxia (GN/GA), the associated dermal and neurological manifestations of CD respectively, may also respond to novel therapies as below.

### SUMMARY OF THE INVENTION

The present invention is directed to methods of treating celiac disease (CD) in a subject. More specifically, methods are presented for treating CD, including non-responsive or slow-responsive CD (NRCD/SRCD) and refractory CD (RCD), with a focus on type II RCD, which has much lower five-year survival rates relative to other types of celiac disease. Additionally, methods are presented for preventing lymphomas that are relatively common in these patients and contribute to the lower five-year survival rates in subjects with Type II RCD.

### BRIEF DESCRIPTION OF THE DRAWINGS

FIG. 1A shows results from a wireless capsule endoscopy of a subject having ulcerative jejunitis, a type II RCD defining condition, prior to tofacitinib administration.

FIG. 1B shows results from a single-balloon enteroscopy of a subject having with ulcerative jejunitis prior to tofacitinib administration.

FIG. 1C shows endoscopic findings during tofacitinib therapy from a patient with Type II RCD who had duodenal nodularity and loss of folds.

FIG. 2A shows results from a jejunum biopsy from a pre-treated patient demonstrating moderate to severe villous atrophy and an increase in intraepithelial lymphocytes, consistent with Marsh 3B-C (hematoxylin and eosin (H&E) staining, 200× original magnification).

FIG. 2B shows an abnormal immunophenotype characterized by expression of CD3, which is compatible with type II RCD FIG. 2C shows an abnormal immunophenotype characterized by the near complete absence of CD8 expression (>90% loss) [normal <50%], which is compatible with type II RCD.

FIG. 2D shows results from a duodenal bulb biopsy from a patient with Type II RCD during tofacitinib therapy (H&E staining, 100× original magnification) showing normal villous architecture with focal areas of crypt hyperplasia and an increase in intraepithelial lymphocytes. The findings are consistent with Marsh 1-2, compatible with resolution of RCD.

### DETAILED DESCRIPTION OF THE INVENTION

The present disclosure relates to methods and compositions for the treatment of celiac disease (CD) and is based, at least in part, on the discovery that administration of tofacitinib, a JAK kinase inhibitor, to a subject having Type II refractory celiac disease (RCD) reversed severe therapy-refractory duodenopathy and ameliorated symptoms of CD. In certain aspects, methods are provided for treating CD and/or preventing progression to lymphoma, comprising administering a Janus kinase (JAK) inhibitor (e.g., tofacitinib or a salt or prodrug thereof) to a subject having CD. Where the present disclosure refers to administering a JAK inhibitor, it should be understood that the JAK inhibitor may likewise be administered as a salt (e.g., a pharmaceutically acceptable salt) thereof, or as a prodrug thereof (or indeed as a salt of such a prodrug), all of which introduce the active ingredient to the patient. For example, when an amount of tofacitinib is given herein, that amount should be understood to reference the amount of tofacitinib as its tofacitinib citrate salt, the drug form currently approved for therapy of ulcerative colitis. If tofacitinib free base is administered, a lesser amount should be administered to account for the weight that the citrate counterion would contribute to a dose of tofacitinib citrate. Similarly, if a different salt or prodrug of tofacitinib is used, the dosage should similarly be adjusted to account for the differing weight of the counterion and/or the weight contributed by the prodrug moiety.

### Definitions

Unless otherwise defined herein, scientific and technical terms used in this application shall have the meanings that are commonly understood by those of ordinary skill in the art. Generally, nomenclature used in connection with, and techniques of, chemistry, cell and tissue culture, molecular biology, cell and cancer biology, neurobiology, neurochemistry, virology, immunology, microbiology, pharmacology, genetics and protein and nucleic acid chemistry, described herein, are those well-known and commonly used in the art.

The methods and techniques of the present disclosure are generally performed, unless otherwise indicated, according to conventional methods well known in the art and as described in various general and more specific references that are cited and discussed throughout this specification See, e.g., “Principles of Neural Science”, McGraw-Hill Medical, New York, N.Y. (2000); Motulsky, “Intuitive Biostatistics”, Oxford University Press, Inc. (1995); Lodish et al., “Molecular Cell Biology, 4th ed.”, W. H. Freeman & Co., New York (2000); Griffiths et al., “Introduction to Genetic Analysis, 7th ed.”, W. H. Freeman & Co., N.Y. (1999); and Gilbert et al., “Developmental Biology, 6th ed.”, Sinauer Associates, Inc., Sunderland, MA (2000).

Chemistry terms used herein, unless otherwise defined herein, are used according to conventional usage in the art, as exemplified by “The McGraw-Hill Dictionary of Chemical Terms,” Parker S., Ed., McGraw-Hill, San Francisco, C.A. (1985).

All of the above, and any other publications, patents and published patent applications referred to in this application are specifically incorporated by reference herein. In case of conflict, the present specification, including its specific definitions, will control.

The term “agent” is used herein to denote a chemical compound (such as an organic or inorganic compound, a mixture of chemical compounds), a biological macromolecule (such as a nucleic acid, an antibody, including parts thereof as well as humanized, chimeric and human antibodies and monoclonal antibodies, a protein or portion thereof, e.g., a peptide, a lipid, a carbohydrate), or an extract made from biological materials such as bacteria, plants, fungi, or animal (particularly mammalian) cells or tissues. Agents include, for example, agents whose structure is known, and those whose structure is not known.

“Administering” or “administration of” a substance, a compound or an agent to a subject can be carried out using one of a variety of methods known to those skilled in the art. For example, a compound or an agent can be administered, intravenously, arterially, intradermally, intramuscularly, intraperitoneally, subcutaneously, ocularly, sublingually, orally (by ingestion), intranasally (by inhalation), intraspinally, intracerebrally, and transdermally (by absorption, e.g., through a skin duct or the rectum). A compound or agent can also appropriately be introduced by rechargeable or biodegradable polymeric devices or other devices, e.g., patches and pumps, or formulations, which provide for the extended, slow or controlled release of the compound or agent. Administering can also be performed, for example, once, a plurality of times, and/or over one or more extended periods.

Appropriate methods of administering a substance, a compound or an agent to a subject will also depend, for example, on the age and/or the physical condition of the subject and the chemical and biological properties of the compound or agent (e.g., solubility, digestibility, bioavailability, stability and toxicity). In some embodiments, a compound or an agent is administered orally, e.g., to a subject by ingestion. In some embodiments, the orally administered compound or agent is in an extended release or slow-release formulation or administered using a device for such slow or extended release.

As used herein, the phrase “conjoint administration” refers to any form of administration of two or more different therapeutic agents such that the second agent is administered while the previously administered therapeutic agent is still effective in the body (e.g., the two agents are simultaneously effective in the patient, which may include synergistic effects of the two agents). For example, the different therapeutic compounds can be administered either in the same formulation or in separate formulations, either concomitantly or sequentially. Thus, an individual who receives such treatment can benefit from a combined effect of different therapeutic agents.

A “therapeutically effective amount” or a “therapeutically effective dose” of a drug or agent is an amount of a drug or an agent that, when administered to a subject will have the intended therapeutic effect. The full therapeutic effect does not necessarily occur by administration of one dose and may occur only after administration of a series of doses. Thus, a therapeutically effective amount may be administered in one or more administrations. The precise effective amount needed for a subject will depend upon, for example, the subject's size, health and age, and the nature and extent of the condition being treated, such as celiac disease. The skilled worker can readily determine the effective amount for a given situation by routine experimentation.

The term “Janus kinase inhibitor” or JAK inhibitor refers to a class of small molecules that can inhibit the activity of one or more JAKs (e.g., JAK1, JAK2, JAK3, TYK2). These inhibitors interfere with the JAK-STAT signaling pathway. Examples of JAK inhibitors include, but are not limited to, ruxolitinib, tofacitinib, peficitinib, oclacitinib, baricitinib, fedratinib, upadacitinib, filgotinib, delgocitinib, abrocitinib, cerdulatinib, gandotinib, lestaurtinib, momelotinib, pacritinib, and deucravacitinib.

The term “modulate” as used herein includes the inhibition or suppression of a function or activity (such as cell proliferation) as well as the enhancement of a function or activity.

The phrase “pharmaceutically acceptable” is art-recognized. In certain embodiments, the term includes compositions, excipients, adjuvants, polymers and other materials and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

A “patient,” “subject,” or “individual” are used interchangeably and refer to either a human or a non-human animal. These terms include mammals, such as humans, primates, livestock animals (including bovines, porcines, etc.), companion animals (e.g., canines, felines, etc.) and rodents (e.g., mice and rats). In some embodiments, the subject is a human who experiences one or more symptoms associated with celiac disease. Such symptoms include but are not limited to abdominal symptoms, loose stools, nausea, and/or gastrointestinal (GI) symptoms. The manifestation of symptoms can occur in those subjects having celiac disease who maintain a gluten-free diet (GFD).

“Pharmaceutically acceptable salt” or “salt” is used herein to refer to an acid addition salt or a basic addition salt which is suitable for or compatible with the treatment of patients. For example, tofacitinib citrate is a pharmaceutically accepted salt of tofacitinib.

By “reduces” is meant a negative alteration of at least 10%, 25%, 50%, 75%, or 100%.

“Treating” a condition or patient refers to taking steps to obtain beneficial or desired results, including clinical results. As used herein, and as well understood in the art, “treatment” is an approach for obtaining beneficial or desired results, including clinical results. Beneficial or desired clinical results can include, but are not limited to, alleviation or amelioration of one or more symptoms or conditions, diminishment of extent of disease, stabilized (i.e., not worsening) state of disease, preventing spread of disease, delay or slowing of disease progression, amelioration or palliation of the disease state, and remission (whether partial or total), whether detectable or undetectable. “Treatment” can also mean prolonging survival as compared to expected survival if not receiving treatment.

“Prodrug” or “pharmaceutically acceptable prodrug” refers to a compound that is metabolized, for example hydrolyzed or oxidized, in the host after administration to form the compound of the present disclosure (e.g., a JAK inhibitor, such as tofacitinib). Typical examples of prodrugs include compounds that have biologically labile or cleavable (protecting) groups on a functional moiety of the active compound. Prodrugs include compounds that can be oxidized, reduced, aminated, deaminated, hydroxylated, dehydroxylated, hydrolyzed, dehydrolyzed, alkylated, dealkylated, acylated, deacylated, phosphorylated, or dephosphorylated to produce the active compound. Examples of prodrugs using ester or phosphoramidate as biologically labile or cleavable (protecting) groups are disclosed in U.S. Pat. Nos. 6,875,751, 7,585,851, and 7,964,580, the disclosures of which are incorporated herein by reference. Such prodrugs are metabolized to produce the active ingredient. The present disclosure includes within its scope the use of prodrugs of JAK kinase inhibitors such as those described in WO2018217699. Conventional procedures for the selection and preparation of suitable prodrugs are described, for example, in “Design of Prodrugs” Ed. H. Bundgaard, Elsevier, 1985.

As used herein, the terms “treat,” treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

As used herein, a therapeutic that “prevents” a disorder or condition refers to a compound that, in a statistical sample, reduces the occurrence of the disorder or condition in the treated sample relative to an untreated control sample, or delays the onset or reduces the severity of one or more symptoms of the disorder or condition relative to the untreated control sample

### Methods of Treatment

The methods and compositions disclosed herein relate to the treatment, prevention, or modulation of celiac disease (CD). CD, as it is generally understood, refers to an autoimmune disease in which a subject with genetic predisposition has an immune response to gluten. Ingestion of gluten by a subject with CD results in an inflammatory cascade with increased intestinal permeability, production of autoantibodies, and small bowel villous atrophy, which can in turn result in impaired nutrient absorption, gastrointestinal and extra-intestinal symptoms, and increased risk of osteoporosis, iron deficiency anemia, infertility, cardiovascular disease, constipation, headaches, myalgia, arthralgia, depression, anxiety, elevated liver enzymes, spleen dysfunction, neuropathy, rashes, foggy mind, and progression to malignancies, among other symptoms and maladies. At present, the only approved therapy for subjects with CD is maintaining a strict gluten-free diet (GFD). Additional experimental therapies are utilized in subjects with refractory celiac disease (RCD), either type I or Type II RCD. “Type I RCD” and “Type II RCD” refer to enteropathy that is non-responsive to a strict GFD for at least 12 months. Ulcerative jejunitis is a rare complication of celiac disease that is usually associated with type II refractory disease. In general, type I RCD responds to stricter dietary modifications and/or corticosteroids, while type II RCD does not and has a much poorer prognosis than type I RCD. For example, type II RCD patients are more likely to develop lymphoma than type I RCD patients, and currently the former is referred to as a pre-lymphoma or low-grade-lymphoma.

CD can be characterized using the modified Marsh classification scheme. The categories used in the modified Marsh classification scheme are shown in Table 1. A subject having a Marsh 0 score would be characterized as either healthy or if previously diagnosed with CD, as “CD in remission”. A Marsh 1 score is non-specific, rarely seen in CD, unless already on a GFD during endoscopy, and may be observed in subjects having dermatitis herpetiformis (DH) or in family members of CD patients, but also with other conditions as infections and adverse response to medications. A Marsh 2 score is rare and is sometimes observed in DH and again in CD patients already on a GFD. Marsh 3 score is characteristic of the spectrum of changes observed in symptomatic CD patients.

TABLE 1
Modified Marsh Classification Scheme

| | IEL/100 | IEL/100 | | |
|---|---|---|---|---|
| Marsh | enterocytes - | enterocytes - | Crypt | |
| Lesion | jejunum | duodenum | hyperplasia | Villi |
| 0 | <40 | <30 | Normal | Normal |
| 1 | >40 | >30 | Normal | Normal |
| 2 | >40 | >30 | Increased | Normal |
| 3a | >40 | >30 | Increased | Partial Atrophy |
| 3b | >40 | >30 | Increased | Subtotal |
| | | | | Atrophy |
| 3c | >40 | >30 | Increased | Total Atrophy |

“IEL” denotes intraepithelial lymphocytes

CD may also be characterized using the villous height to crypt depth (Vh:Cd) ratio. A normal Vh:Cd ratio is above 3:1. Lower ratios are indicative of a flattening of the small bowel, intestinal injury, and a deteriorating clinical status. Though variable, Vh:Cd ratio of less than 3:1 is suggestive of active CD.

One aspect of the present disclosure provides a method of treating CD by administering a JAK inhibitor (e.g., ruxolitinib, tofacitinib, peficitinib, oclacitinib, baricitinib, fedratinib, upadacitinib, filgotinib, delgocitinib, abrocitinib, cerdulatinib, gandotinib, lestaurtinib, momelotinib, pacritinib, or deucravacitinib, preferably tofacitinib) to a subject. The subject to be treated with the JAK inhibitor preferably has refractory celiac disease (RCD), most preferably Type II RCD. In certain aspects, the present disclosure provides methods of reducing one or more symptoms or associated complications of CD (e.g., RCD) in a subject by administering a JAK inhibitor to a subject having or suspected of having CD. In some embodiments, the JAK inhibitor is tofaticinib or ruxolitinib (see Nandi et al. (2022) Clinics and Research in Hepatology and Gastroenterology (2022), doi.org/10.1016/j.clinre.2022.101960, available online May 24, 2022 and the contents of which are incorporated herein by reference). In some embodiments, the subject has ulcerative jejunitis, a rare complication of celiac disease. In yet another aspect, a method is provided for treating ulcerative jejunitis by administering a JAK inhibitor (e.g., tofacitinib or ruxolitinib) to a subject.

Another aspect of the present disclosure provides methods for reducing, ameliorating, or otherwise lessening a symptom of CD by administering a JAK inhibitor to the subject. “Steatorrhea,” a symptom often observed in subjects diagnosed with CD, refers to increased fat in feces that cause anal leakage and/or fecal incontinence and malabsorption. Another symptom associated with CD is diarrhea, which can be accompanied by increased abdominal pain, cramping, distention, and/or bloating. CD subjects can become lactose intolerant and/or experience weight loss. They can develop osteoporosis and/or anemia, which results when the small intestines cannot absorb sufficient amounts of nutrients including, but not limited to, iron, folate, vitamin D or vitamin B12. Other symptoms or signs of CD include, but are not limited to, constipation, headaches, myalgia, arthralgia, depression, anxiety, infertility, elevated liver enzymes, spleen dysfunction, neuropathy, rashes, and foggy mind. Symptoms can vary from subject to subject; thus, samples obtained from the patient at different times during treatment can be analyzed to determine the effect of the JAK inhibitor treatment on the subject's CD. For example, lesions can be characterized using the modified Marsh classification system to detect any changes in the intestinal damage in a subject having CD.

A JAK inhibitor (e.g., tofacitinib) may be useful and even curative also in patients with uncomplicated CD, non-responsive or slow-responsive CD (NRCD/SRCD), and type I RCD, as a substitute for or adjunct to the mandatory strict GFD, accelerating recovery and preventing associated neoplastic and malabsorptive complications. Many patients struggle with adhering to GFD and food can be inadvertently cross-contaminated with gluten when food is prepared (e.g., in restaurants or at work). The drug can be used not only to treat RCD patients but also those with CD in remission as a substitute or alternative to a strict GFD. In some embodiments, a CD patient is able to tolerate greater than 20 ppm of gluten when administered a JAK inhibitor (e.g., tofacitinib or ruxolitinib). Dermatitis herpetiformis (DH) and gluten neuropathy/ataxia (GN/GA), the associated dermal and neurological manifestations of CD, may also respond to this novel therapy.

In the methods disclosed herein, a JAK inhibitor (e.g., tofacitinib or ruxolitinib) can be conjointly administered with an additional agent and/or a gluten free diet or may substitute the latter. The additional agent and the JAK inhibitor can be used to treat a subject's CD and/or reduce at least one symptom/manifestation of the subject's CD. In some embodiments, the efficacy of the conjoint therapy can be assessed in the same manner as the JAK inhibitor-only therapy as described above (i.e., assaying samples obtained from a subject having CD at different time points, e.g., prior to and post administration of the one or more of the agents in the combination therapy). In some embodiments, the JAK inhibitor and the additional agent are administered simultaneously or sequentially, while in other embodiments, the tofacitinib and the additional agent are administered at different times.

### Screening Methods

One aspect of the present invention relates to screening assays that identify if a subject's CD is likely to respond to a JAK inhibitor (e.g., tofacitinib or ruxolitinib) administration. Screening assays may also be used to identify agents, in combination with a JAK inhibitor, that treat, prevent, or otherwise modulate (e.g., reduce symptoms/manifestations) of CD. Identifying such an agent involves determining the ability of the agent to treat, prevent, or otherwise modulate CD, for example, by monitoring the severity, progression, development, reduction, or elimination of a subject's symptoms/signs. Methods for visualizing a subject's small intestines may be used to determine the status of a subject's CD and are known in the art (e.g., esophagogastroduodenoscopy, enteroscopy, capsule endoscopy, radiographic imaging). Additional blood and/or tissue samples can be used to determine titers of celiac autoantibodies and also the relative of abundance of intraepithelial lymphocytes compared to enterocytes in a subject having or suspected of having CD (e.g., in the subject's small bowel).

The effectiveness of treating a subject's CD with a JAK inhibitor can be assessed using any method known in the art. For example, the presence and/or severity of a subject's CD can be determined at a first time point (e.g., prior to administration of a JAK inhibitor) and at a second time point (e.g., during or post-administration of a JAK inhibitor). Detecting the presence and/or determining the severity of a subject's CD can be accomplished by using any number of techniques to assess standard criteria. Such techniques include, but are not limited to, endoscopic examination, small bowel imaging, immunohistochemistry, flow cytometry, blood and tissue sample analysis, and molecular genetics. Additionally, immunoassays, PCR (e.g., RT-PCR and qPCR), chromosomal analysis, biomarker analysis, and physical examination of a subject can be used in assessing a subject.

A JAK inhibitor (e.g., tofacitinib or ruxolitinib) can be administered to a subject having CD in any form, although it is often formulated as a tablet allowing for non-invasive oral administration. Other means of administration are contemplated herein. For example, administration may be accomplished by parenteral, intravenous, intra-arterial, subcutaneous, intramuscular, intraventricular, by aerosol, by suppositories, or by intranasal administration. In some embodiments, between about 1 mg and about 50 mg; between about 1 mg and about 25 mg, between about 1 mg and about 10 mg, and between about 1 mg and 5 mg of a JAK inhibitor is administered to a subject having or suspected of having CD. In some embodiments, between about 5 mg and about 50 mg, between about 10 mg and about 50 mg, or between about 25 and about 50 mg of a JAK inhibitor is administered to a subject having or suspected of having CD. A JAK inhibitor can be administered one or more times a day. For example, a subject may be administered a JAK inhibitor one, two, three, or even four times a day. In certain preferred embodiments, the JAK inhibitor is administered twice daily in 10-mg doses or 5-mg doses, e.g., depending on the severity of the condition and the patient's response to the initial dosage. In some embodiments, the JAK inhibitor is administered in multiple equal doses.

The present invention also pertains to monitoring the influence of a JAK inhibitor, alone or in combination with one or more additional therapeutic agents, on CD. For example, monitoring the influence of a JAK inhibitor on a subject's CD can comprise clinical response and/or visualization of the subject's intestinal tract (e.g., esophagogastroduodenoscopy (EGD)) during a course of treatment to determine changes in intestinal damage. In some embodiments, a first image of the intestines is acquired at or near the time treatment is commenced, and the intestinal damage observed determined from this first image serves as a reference to which later-acquired images can be compared. In some embodiments, intestinal lesions are characterized using the modified Marsh classification system or villous-height to crypt depth ratio (Vh:Cd). A reduction in enteropathy or intestinal damage (i.e., improvement of Marsh lesions) during the course of treatment indicates a positive therapeutic response to treatment.

### Pharmaceutical Compositions

The compositions and methods of the present invention may be utilized to treat an individual in need thereof. In certain embodiments, the individual is a mammal such as a human, or a non-human mammal. When administered to an animal, such as a human, the composition or the compound is preferably administered as a pharmaceutical composition comprising, for example, a compound of the invention and a pharmaceutically acceptable carrier. Pharmaceutically acceptable carriers are well known in the art and include, for example, aqueous solutions such as water or physiologically buffered saline or other solvents or vehicles such as glycols, glycerol, oils such as olive oil, or injectable organic esters. In preferred embodiments, when such pharmaceutical compositions are for human administration, particularly for invasive routes of administration (i.e., routes, such as injection or implantation, that circumvent transport or diffusion through an epithelial barrier), the aqueous solution is pyrogen-free, or substantially pyrogen-free. The excipients can be chosen, for example, to effect delayed release of an agent or to selectively target one or more cells, tissues, or organs. The pharmaceutical composition can be in dosage unit form such as tablet, capsule (including sprinkle capsule and gelatin capsule), granule, lyophile for reconstitution, powder, solution, syrup, suppository, injection or the like. The composition can also be present in a transdermal delivery system, e.g., a skin patch. The composition can also be present in a solution suitable for topical administration, such as a lotion, cream, or ointment.

The phrase “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the patient. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

A pharmaceutically acceptable carrier can contain physiologically acceptable agents that act, for example, to stabilize, increase solubility or to increase the absorption of a compound such as a JAK inhibitor (e.g., tofacitinib or ruxolitinib). Such physiologically acceptable agents include, for example, carbohydrates, such as glucose, sucrose or dextrans, antioxidants, such as ascorbic acid or glutathione, chelating agents, low molecular weight proteins or other stabilizers or excipients. The choice of a pharmaceutically acceptable carrier, including a physiologically acceptable agent, depends, for example, on the route of administration of the composition. The preparation or pharmaceutical composition can be a self-emulsifying drug delivery system or a self-microemulsifying drug delivery system. The pharmaceutical composition (preparation) also can be a liposome or other polymer matrix, which can have incorporated therein, for example, a compound of the invention. Liposomes, for example, which comprise phospholipids or other lipids, are nontoxic, physiologically acceptable and metabolizable carriers that are relatively simple to make and administer.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

A pharmaceutical composition (preparation) can be administered to a subject by any of a number of routes of administration including, for example, orally (for example, drenches as in aqueous or non-aqueous solutions or suspensions, tablets, capsules (including sprinkle capsules and gelatin capsules), boluses, powders, granules, pastes for application to the tongue); absorption through the oral mucosa (e.g., sublingually); subcutaneously; transdermally (for example as a patch applied to the skin); and topically (for example, as a cream, ointment or spray applied to the skin). The compound may also be formulated for inhalation. In certain embodiments, a compound may be simply dissolved or suspended in sterile water. Details of appropriate routes of administration and compositions suitable for same can be found in, for example, U.S. Pat. Nos. 6,110,973, 5,763,493, 5,731,000, 5,541,231, 5,427,798, 5,358,970 and 4,172,896, as well as in patents cited therein.

The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient that can be combined with a carrier material to produce a single dosage form will generally be that amount of the compound that produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association an active compound, such as a JAK inhibitor, with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules (including sprinkle capsules and gelatin capsules), cachets, pills, tablets, lozenges (using a flavored basis, usually sucrose and acacia or tragacanth), lyophile, powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. Compositions or compounds may also be administered as a bolus, electuary or paste.

To prepare solid dosage forms for oral administration (capsules (including sprinkle capsules and gelatin capsules), tablets, pills, dragees, powders, granules and the like), the active ingredient is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof, (10) complexing agents, such as, modified and unmodified cyclodextrins; and (11) coloring agents. In the case of capsules (including sprinkle capsules and gelatin capsules), tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of pharmaceutical compositions, such as dragees, capsules (including sprinkle capsules and gelatin capsules), pills and granules, may optionally be scored or prepared with coatings and shells, such as enteric coatings and other coatings well known in the pharmaceutical-formulating art They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions that can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions that can be used include polymeric substances and waxes. The active ingredient can also be in micro-encapsulated form, if appropriate, with one or more of the above-described excipients.

Liquid dosage forms useful for oral administration include pharmaceutically acceptable emulsions, lyophiles for reconstitution, microemulsions, solutions, suspensions, syrups and elixirs. In addition to the active ingredient, the liquid dosage forms may contain inert diluents commonly used in the art, such as, for example, water or other solvents, cyclodextrins and derivatives thereof, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion. Pharmaceutical compositions suitable for parenteral administration comprise one or more active compounds in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers that may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents that delay absorption such as aluminum monostearate and gelatin.

The subject receiving this treatment is any animal in need, including primates, in particular humans, and animal models of celiac disease.

In certain embodiments, compounds of the invention may be used alone or conjointly administered with another type of therapeutic agent.

The present disclosure includes the use of pharmaceutically acceptable salts of a JAK inhibitor (e.g., tofacitinib or ruxolitinib) in the compositions and methods of the present invention. In certain embodiments, contemplated salts include, but are not limited to, 1-hydroxy-2-naphthoic acid, 2,2-dichloroacetic acid, 2-hydroxyethanesulfonic acid, 2-oxoglutaric acid, 4-acetamidobenzoic acid, 4-aminosalicylic acid, acetic acid, adipic acid, 1-ascorbic acid, l-aspartic acid, benzenesulfonic acid, benzoic acid, (+)-camphoric acid, (+)-camphor-10-sulfonic acid, capric acid (decanoic acid), caproic acid (hexanoic acid), caprylic acid (octanoic acid), carbonic acid, cinnamic acid, citric acid, cyclamic acid, dodecylsulfuric acid, ethane-1,2-disulfonic acid, ethanesulfonic acid, formic acid, fumaric acid, galactaric acid, gentisic acid, d-glucoheptonic acid, d-gluconic acid, d-glucuronic acid, glutamic acid, glutaric acid, glycerophosphoric acid, glycolic acid, hippuric acid, hydrobromic acid, hydrochloric acid, isobutyric acid, lactic acid, lactobionic acid, lauric acid, maleic acid, 1-malic acid, malonic acid, mandelic acid, methanesulfonic acid, naphthalene-1,5-disulfonic acid, naphthalene-2-sulfonic acid, nicotinic acid, nitric acid, oleic acid, oxalic acid, palmitic acid, pamoic acid, phosphoric acid, proprionic acid, 1-pyroglutamic acid, salicylic acid, sebacic acid, stearic acid, succinic acid, sulfuric acid, 1-tartaric acid, thiocyanic acid, p-toluenesulfonic acid, trifluoroacetic acid, and undecylenic acid salts.

### EXAMPLES

The invention now being generally described, it will be more readily understood by reference to the following examples which are included merely for purposes of illustration of certain aspects and embodiments of the present invention and are not intended to limit the invention.

### Example 1: Successful Use of Tofacitinib for Treating Type II Refractory Celiac Disease

A 44-year-old female with a past medical history significant for Hashimoto's thyroiditis and positive celiac serology was initially seen for protein-losing enteropathy. Although she had previously been on a gluten-free diet (GFD) for 1 year after first being diagnosed with CD, she re-introduced gluten 3 years before pregnancy without symptoms. She stayed on gluten until one-month postpartum when she developed epigastric pain, nausea, diarrhea with 2-3 loose to watery stools daily, along with significant bilateral lower extremity edema.

At the time of initial hospital admission, she was five months postpartum with the above symptomatology. Esophagogastroduodenoscopy (EGD) showed marked flattening of duodenal villi and scalloping suggestive of CD and biopsies confirmed Marsh 3B lesions. She was discharged to outpatient setting on a GFD and referred to a CD clinic for management.

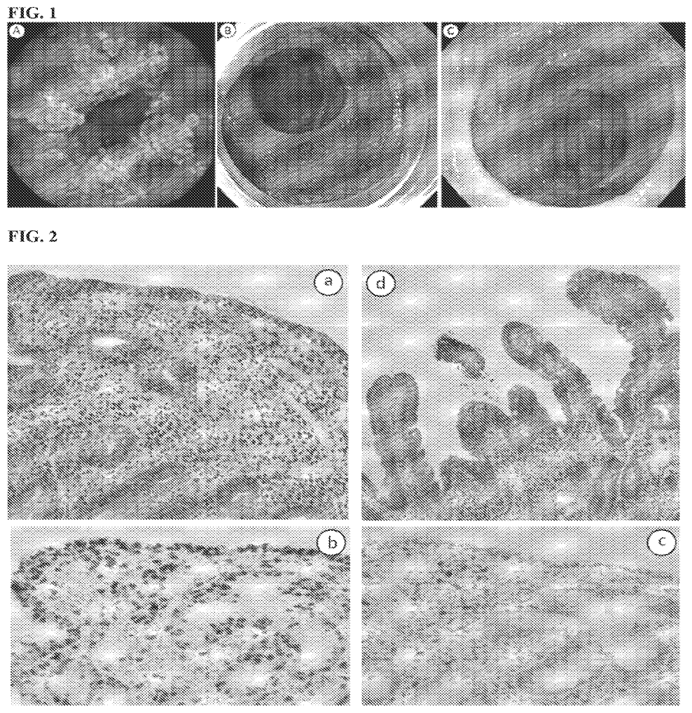

During the initial outpatient visit in the CD clinic she was hypotensive with anasarca, diagnosed with celiac crisis, and urgently sent for readmission. She was treated with 72 hours of intravenous solumedrol 20 mg every 12 hours and then transitioned to open-capsule protocol budesonide 3 mg three times a day. During that admission she underwent capsule endoscopy which showed grossly abnormal small bowel with diffuse ulcerations, oozing of blood, mosaicism, diffuse villous blunting and a small bowel structure (FIG. 1A). These findings were followed promptly with a single balloon enteroscopy with biopsies She was subsequently diagnosed with Type II RCD with ulcerative jejunitis (FIG. 1B) and the repeat biopsies confirmed Marsh 3B-C lesions (FIGS. 2A-2C) with T-cell receptor gamma (TRG) locus gene rearrangement demonstrating abnormal clonality.

Enteroscopy after the patient was on budesonide for 3 months showed steroid-resistant RCD and ulcerative jejunitis with persistent Marsh 3B-C lesions. Additionally, she continued to have marked loss of CDS expression in intraepithelial T-lymphocytes, supporting the diagnosis of Type II RCD. She was therefore switched to experimental infliximab after a total of 5 months on budesonide. She was induced with infliximab and was on maintenance for a total of 9 months with resolution of her ulcerative jejunitis but only a modest histological improvement to Marsh 3A, with unchanged T-cell aberrancy.

Due to corticosteroid- and anti-TNF-refractory disease, she was referred to oncology for a trial of one dose of cladribine 10 mg IV, unfortunately without resolution of her RCD, so a second dose was not administered. The patient was now about 18 months out from diagnosis of Type II RCD, which is associated with about 50% risk of lymphoma in 5 years, so it was imperative to find an effective regimen. In light of positive yet statistically insignificant results of AMG714, anti-IL-15 mAb, off-label use of tofacitinib was started, an inhibitor of Janus kinase (JAK) enzymes with associated IL-15 inhibition, at 10 mg twice daily. Discussion of associated adverse events, including thromboembolic ones, was conducted, and pre-treatment labs including lipid profile were done. Four months after the start of tofacitinib therapy the patient had repeat upper endoscopy, which showed improvement in her duodenopathy (FIG. 1C) with biopsies confirming down-staging of her disease to Marsh 1-2 lesions (FIG. 2C) and normalization of her CD serologies (Table 2). This was the first-time the patient achieved such results in the 2 years since presenting with celiac crisis. Along with endoscopic, serological, and immunohistochemical improvement, she reported feeling well with minimum symptoms. Her tofacitinib dose will be reduced to 5 mg twice daily to reduce associated adverse events.

TABLE 2
Trend of pertinent laboratory values and histology throughout her treatments

| | Initial | | Celiac | | After | | After | After | During |
|---|---|---|---|---|---|---|---|---|---|
| | admission | | Crisis | | Budesonide | | Infliximab | Cladribine | Tofacitinib |
| | Time 0 | | Time 2 wk | | Time 3 m | | Time 1 y | Time 1.5 y | Time 2 y |
| WBC | 5.0 | | 2.7 | (L) | 7.5 | | 7.1 | 4.2 | 3.2 |
| Hemoglobin | 10.7 | (L) | 10.9 | (L) | 12.1 | | 11.7 | 13.2 | 12.0 |
| Platelets | 734 | (H) | 539 | (H) | 707 | (H) | 298 | 362 | 373 |
| CRP | 1.2 | (H) | <0.3 | | <0.3 | | <0.3 | <0.3 | <0.3 |
| DGP IgA | 237.7 | (H) | 205.2 | (H) | 186.9 | (H) | 53.9 (H) | 57.7 (H) | 70 (H) |
| TTG IgA | 300.5 | (H) | 206.0 | (H) | 152.3 | (H) | 33.1 (H) | 44.3 (H) | 5 |

| | | | | | | |
|---|---|---|---|---|---|---|
| Endomysial IgA | <1:10 | Positive | Positive | <1:10 | <1:10 | <1:10 |
| Pathology | Marsh 3B | Marsh 3B-C | Marsh 3B-C | Marsh 3A | Marsh 3A | Marsh 1-2 |

The exact prevalence of RCD is not well known, with the most reliable data suggesting the rate is less than 1.5% of all Celiac patients and less than one percent having specifically Type II RCD6. However, malabsorption, malignancy, morbidity and mortality are known complications in Type II RCD (Cellier et al. (2000); Ilus et al. (2014); Weiss (2021) Diagnosis and Management of Gluten-Associated Disorders: A clinical casebook. Springer). Currently, the best clinical practice in managing RCD is to advocate for experimental medication treatment in adjunct to a strict gluten-free diet (GFD). Yet the same guidelines acknowledge the absence of treatments with proven efficacy in Type II RCD (Rubio-Tapia et al. (2013) Am. J. Gastroenterol., 108(5):656-677; Al-Toma et al. (2019) United European Gastroenterol. J.: 7(5):583-613). The European guidelines discuss the potential use of AMG714 or JAK inhibitor after corticosteroids, immunomodulators, and chemotherapy in the most refractory of CD cases. The present study showed the successful use of a JAK enzyme inhibitor after several set wise failures in the treatment of Type II RC and no associated adverse events. The novel use of this agent shows promise to reverse this pre-lymphoma state. Additional studies are needed, but the therapy disclosed herein may be utilized in uncomplicated CD or non-responsive CD (NRCD) in combination with a GFD, or even as a substitute to one.

### INCORPORATION BY REFERENCE

All publications and patents mentioned herein are hereby incorporated by reference in their entirety as if each individual publication or patent was specifically and individually indicated to be incorporated by reference. In case of conflict, the present application, including any definitions herein, will control.

### EQUIVALENTS

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

We claim:

1. A method for treating celiac disease (CD) in a subject, the method comprising administering a JAK inhibitor, or a salt or prodrug thereof, to the subject.

2. A method for reducing at least one symptom/sign of celiac disease (CD) in a subject, the method comprising administering a JAK inhibitor, or a salt or prodrug thereof, to the subject.

3. The method of claim 1 or 2, wherein the subject also has or is suspected of having ulcerative jejunitis.

4. A method of treating ulcerative jejunitis in a subject, the method comprising administering a JAK inhibitor, or a salt or prodrug thereof, to the subject.

5. A method for reducing at least one symptom/sign of ulcerative jejunitis in a subject, the method comprising administering a JAK inhibitor, or a salt or prodrug thereof, to the subject.

6. The method of any one of claims 1-5, further comprising administering one or more additional agents.

7. The method of any one of claims 1-5, wherein the JAK inhibitor is ruxolitinib, tofacitinib, peficitinib, oclacitinib, baricitinib, fedratinib, upadacitinib, filgotinib, delgocitinib, abrocitinib, cerdulatinib, gandotinib, lestaurtinib, momelotinib, pacritinib, or deucravacitinib, preferably tofacitinib.

8. The method of claim 7, wherein administering tofacitinib comprises administering between about 1 mg and about 50 mg of tofacitinib.

9. The method of claim 8, wherein administering tofacitinib comprises administering about 10 mg of tofacitinib.

10. The method of any one of claims 7-9, wherein the tofacitinib is tofacitinib citrate.

11. The method of any one of claims 1-10, wherein the JAK inhibitor is administered from one to four times per day.

12. The method of claim 12, wherein the JAK inhibitor is administered once or twice per day.

13. The method of any one of claims 1-12, wherein the JAK inhibitor is formulated in a tablet.

14. The method of any one of claims 1-13, wherein the JAK inhibitor is administered orally.

15. The method of any one of claims 1-14, wherein the subject has type II refractory CD.

16. The method of any one of claims 1-14, wherein the subject has type I RCD.

17. The method of any one of claims 1-14, wherein the subject has non-responsive CD.

18. The method of any one of claims 1-14, wherein the subject has dermatitis herpetiformis.

19. The method of any one of claims 1-14, wherein the subject has gluten neuropathy and/or gluten ataxia.

20. The method of claim 2 or 5, wherein the symptom/sign is steatorrhea, diarrhea, abdominal pain, weight loss, anemia, vitamin/mineral deficiency, loss of bone density, constipation, headaches, myalgia, arthralgia, depression, anxiety, infertility, elevated liver enzymes, spleen dysfunction, neuropathy, rashes, and/or foggy mind.

21. The method of any one of claims 1-20, further comprising assessing the efficacy of the JAK inhibitor.

22. The method of claim 21, wherein assessing the efficacy of the JAK inhibitor to treat comprises:
obtaining a first subject sample;
characterizing the subject's CD;
obtaining a second subject sample, wherein the second subject sample is obtained from the subject at a later time than when the first subject sample was obtained from the subject;
characterizing the subject's CD at the later time point; and
comparing the subject's CD at the first time point to the subject's CD at the second time point, thereby assessing the efficacy of the JAK inhibitor.

23. The method of claim 22, wherein the first and second subject samples are the same type of sample.

24. The method of claim 22 or 23, wherein the first and second subject samples are blood samples, tissue biopsies, or both.

25. The method of claim 22 or 23, wherein the first and second subject samples are images of a portion of the subject's small intestine.

26. The method of claim 22, wherein characterizing the subject's CD comprises determining the number of intraepithelial lymphocytes per 100 enterocytes in the small bowel.

27. The method of claim 26, wherein the subject has more than 40 intraepithelial lymphocytes per 100 enterocytes in the jejunum.

28. The method of claim 26 or 27, wherein the subject has more than 30 intraepithelial lymphocytes per 100 enterocytes in the duodenum.

29. The method of claim 22, wherein the subject has partial, subtotal or total atrophy of the villi in the small intestine.

30. The method of any one of claims 22-39, wherein the subject has lesions in the small intestines that are 3a, 3b, or 3c on the modified Marsh scale.

31. The method of any one of claims 22-29, wherein the subject has a villous height-to crypt depth ratio of less than 3.

32. A method of preventing lymphoma in a subject having or suspected of having celiac disease (CD), comprising administering a JAK inhibitor, or a salt or prodrug thereof, to the subject.

33. A method of reducing the risk of lymphoma in a subject having or suspected of having celiac disease (CD), comprising administering a JAK inhibitor, or a salt or prodrug thereof, to the subject.

34. The method of claim 32 or 33, wherein the JAK inhibitor is ruxolitinib, tofacitinib, peficitinib, oclacitinib, baricitinib, fedratinib, upadacitinib, filgotinib, delgocitinib, abrocitinib, cerdulatinib, gandotinib, lestaurtinib, momelotinib, pacritinib, or deucravacitinib, preferably tofacitinib.

35. The method of claim 34, wherein administering tofacitinib comprises between about 1 mg and about 50 mg of tofacitinib.

36. The method of claim 35, wherein administering tofacitinib comprises about 10 mg of tofacitinib.

37. The method of any one of claims 34-36, wherein the tofacitinib is tofacitinib citrate.

38. The method of any one of claims 34-37, wherein the tofacitinib, or salt or prodrug thereof, is administered from one to four times per day.

39. The method of claim 38, wherein the tofacitinib, or a salt or prodrug thereof, is administered once or twice per day.

40. The method of any one of claims 34-39, wherein the JAK inhibitor formulated in a tablet.

41. The method of any one of claims 34-40, further comprising assessing the efficacy of the JAK inhibitor.

42. The method of claim 41, wherein assessing the efficacy of a JAK inhibitor to treat comprises:
obtaining a first subject sample;
characterizing the subject's CD;
obtaining a second subject sample, wherein the second subject sample is obtained from the subject at a later time than when the first subject sample was obtained from the subject;
characterizing the subject's CD at the later time point; and
comparing the subject's CD at the first time point to the subject's CD at the second time point, thereby assessing the efficacy of the JAK inhibitor.

43. The method of claim 42, wherein the first and second subject samples are the same type of sample.

44. The method of claim 42 or 43, wherein the first and second subject samples are blood samples, tissue biopsies, or both.

45. The method of claim 42 or 43, wherein the first and second subject samples are images of a portion of the subject's small intestine.

46. The method of any one of claims 42-45, wherein the subject has lesions in the small intestines that are 3a, 3b, or 3c on the modified Marsh scale.

47. The method of any one of claims 42-45, wherein the subject has a villous height-to crypt depth ratio of less than 3.
